(19) United States
(12) Patent Application Publication
(10) Pub. No.: US 2025/0359728 A1
(43) Pub. Date: Nov. 27, 2025

(54) CONTROL DEVICE, MEDICAL SYSTEM, OPERATION METHOD OF MEDICAL DEVICE, AND COMPUTER-READABLE RECORDING MEDIUM

(71) Applicant: OLYMPUS MEDICAL SYSTEMS CORP., Tokyo (JP)

(72) Inventors: Yasuo TANIGAMI, Tokyo (JP); Yusuke OTSUKA, Yokohama-shi (JP); Noriko KURODA, Tokyo (JP); Takaaki IGARASHI, Tokyo (JP); Maho NISHIYAMA, Tokyo (JP)

(73) Assignee: OLYMPUS MEDICAL SYSTEMS CORP., Tokyo (JP)

(21) Appl. No.: 19/292,059

(22) Filed: Aug. 6, 2025

Related U.S. Application Data
(63) Continuation of application No. PCT/JP2023/004397, filed on Feb. 9, 2023.

(51) Int. Cl. A61B 1/00; A61B 1/04; A61B 1/06; G06T 7/00; G06T 7/12; G16H 30/40

(52) U.S. Cl. CPC A61B 1/00009; A61B 1/00045; A61B 1/043; A61B 1/0661; G06T 7/0012; G06T 7/12; G16H 30/40; G06T 2207/10064; G06T 2207/10068; G06T 2207/30096

(57) ABSTRACT

A control device for use with a medical device includes a processor includes hardware. The processor is configured to: extract a first thermal treatment region treated by an energy device from a first fluorescence image for a biological tissue; impart information of an output mode of the energy device to the first thermal treatment region of the fluorescence image; and output the information of the output mode imparted to the first thermal treatment region.

### CROSS REFERENCES TO RELATED APPLICATIONS

This application is a continuation of International Application No. PCT/JP2023/004397, filed on Feb. 9, 2023, the entire contents of which are incorporated herein by reference.

### BACKGROUND

### 1. Technical Field

The present disclosure relates to a control device, a medical system, an operation method of the medical device, and a computer-readable recording medium that perform image processing on an imaging signal obtained by imaging a subject and output the imaging signal.

### 2. Related Art

In the related art, there is known a technique in which a surgical endoscope is inserted into a subject, and a biological tissue is cauterized and treated by a treatment tool such as an energy device while an operator observes a treatment portion (for example, refer to WO 2020/174666 A).

Here, when a biological tissue is cauterized, advanced glycation end-products (AGEs), so-called “scorches” occur due to thermal denaturation. This AGEs emits fluorescence by light of a specific wavelength. The operator can confirm a thermally denatured region of the treatment portion by observing an image of the fluorescence emitted by the AGEs.

### SUMMARY

In some embodiments, a control device for use with a medical device includes a processor includes hardware. The processor is configured to: extract a first thermal treatment region treated by an energy device from a first fluorescence image for a biological tissue; impart information of an output mode of the energy device to the first thermal treatment region of the fluorescence image; and output the information of the output mode imparted to the first thermal treatment region.

In some embodiments, a medical system includes: an imaging device configured to capture an image of a subject; a light source device configured to emit excitation light that excites a substance generated by applying a thermal treatment to a biological tissue; and the control device. The control device is electrically connected to an imaging device and configured to communicate with a device control device that controls an energy device that cauterizes a treatment target.

In some embodiments, provided is an operation method of a medical device. The operation method includes: extracting a thermal treatment region treated by an energy device from a fluorescence image for a biological tissue; imparting information of an output mode of the energy device to the thermal treatment region of the fluorescence image; and outputting the information of the output mode imparted to the thermal treatment region.

In some embodiments, provided is a non-transitory computer-readable recording medium with an executable program stored thereon. The program causes a medical device to execute: extracting a thermal treatment region treated by an energy device from the fluorescence image for a biological tissue; imparting information of an output mode of the energy device to the thermal treatment region of the fluorescence image; and outputting the information of the output mode is imparted to the thermal treatment region.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a schematic configuration of an endoscope system according to a first embodiment;

FIG. 2 is a diagram illustrating a schematic configuration of a treatment system connected to the endoscope system according to the first embodiment;

FIG. 3 is a block diagram illustrating a functional configuration of a main part of the endoscope system according to the first embodiment;

FIG. 4 is a diagram schematically illustrating wavelength characteristics of light emitted by first and second light source units according to the first embodiment;

FIG. 5 is a diagram schematically illustrating a configuration of a pixel unit according to the first embodiment;

FIG. 6 is a diagram schematically illustrating a configuration of a color filter according to the first embodiment;

FIG. 7 is a diagram schematically illustrating sensitivity characteristics of each filter;

FIG. 8A is a diagram schematically illustrating signal values of G pixels of an imaging element according to the first embodiment;

FIG. 8B is a diagram schematically illustrating signal values of R pixels of the imaging element according to the first embodiment;

FIG. 8C is a diagram schematically illustrating signal values of B pixels of the imaging element according to the first embodiment;

FIG. 9 is a diagram schematically illustrating a configuration of a cut filter according to the first embodiment;

FIG. 10 is a diagram schematically illustrating transmission characteristics of the cut filter according to the first embodiment;

FIG. 11 is a diagram schematically illustrating an observation principle in a normal light observation mode according to the first embodiment;

FIG. 12 is a diagram schematically illustrating an observation principle in a thermal treatment observation mode according to the first embodiment;

FIG. 13 is a flowchart for description of thermally denatured region determination processing using the endoscope system according to the first embodiment;

FIG. 14 is a diagram for description of a fluorescence shape in a fluorescence observation mode;

FIG. 15 is a diagram illustrating a schematic configuration of an endoscope system according to a second embodiment;

FIG. 16 is a block diagram illustrating a functional configuration of a main part of the endoscope system according to the second embodiment; and

FIG. 17 is a diagram illustrating a schematic configuration of a surgical microscope system according to a third embodiment.

### DETAILED DESCRIPTION

Hereinafter, modes for carrying out the present disclosure will be described in detail with reference to the drawings. Note that the present disclosure is not limited to the following embodiments. In addition, each drawing referred to in the following description merely schematically illustrates a shape, a size, and a positional relationship to an extent that a content of the present disclosure can be understood. That is, the present disclosure is not limited only to the shape, the size, and the positional relationship illustrated in each drawing. Furthermore, in the description of the drawings, the same portions will be denoted by the same reference numerals. Furthermore, as an example of an endoscope system according to the present disclosure, an endoscope system including a rigid endoscope and a medical imaging device will be described.

### First Embodiment

### Configuration of Endoscope System

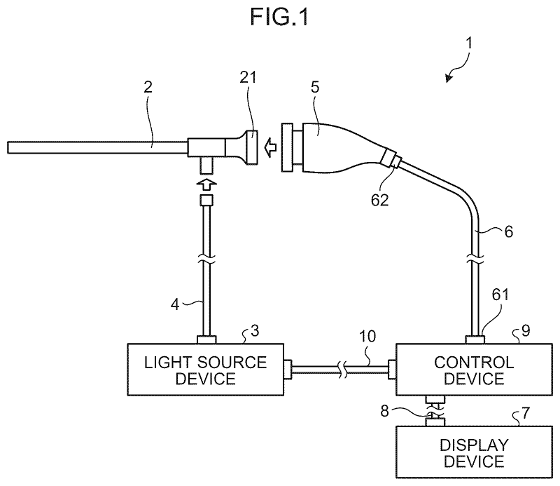

FIG. 1 is a diagram illustrating a schematic configuration of an endoscope system according to a first embodiment. An endoscope system 1 illustrated in FIG. 1 is a system that is used in a medical field and observes a biological tissue in a subject such as a living body. The endoscope system 1 is used when a subject is operated or treated using a treatment tool such as an energy device capable of performing thermal treatment. An operator performs surgery, treatment, or the like while observing a display device on which an observation image based on image data captured by a medical imaging device is displayed.

The endoscope system 1 includes an insertion unit 2, a light source device 3, a light guide 4, an endoscope camera head 5 (medical imaging device), a first transmission cable 6, a display device 7, a second transmission cable 8, a control device 9, and a third transmission cable 10.

The insertion unit 2 is rigid or at least partially flexible and has an elongated shape. The insertion unit 2 is inserted into a subject such as a patient via a trocar. The insertion unit 2 is provided with an optical system such as a lens that forms an observation image therein.

The light source device 3 is connected to one end of the light guide 4, and supplies illumination light to irradiate the inside of the subject to one end of the light guide 4 under the control of the control device 9. The light source device 3 is realized by using one or more light sources of a light emitting diode (LED) light source, a xenon lamp, and a semiconductor laser element such as a laser diode (LD), a processor which is a processing device having hardware such as a field programmable gate array (FPGA) and a central processing unit (CPU), and a memory which is a temporary storage area used by the processor.

One end of the light guide 4 is detachably connected to the light source device 3, and the other end thereof is detachably connected to the insertion unit 2. The light guide 4 guides illumination light supplied from the light source device 3 from one end to the other end and supplies the illumination light to the insertion unit 2.

An eyepiece unit 21 of the insertion unit 2 is detachably connected to the endoscope camera head 5. Under the control of the control device 9, the endoscope camera head 5 generates an imaging signal (RAW data) by receiving an observation image formed by the insertion unit 2 and performing photoelectric conversion, and outputs the imaging signal to the control device 9 via the first transmission cable 6.

One end of the first transmission cable 6 is detachably connected to the control device 9 via a video connector 61, and the other end thereof is detachably connected to the endoscope camera head 5 via a camera head connector 62. The first transmission cable 6 transmits the imaging signal output from the endoscope camera head 5 to the control device 9, and transmits setting data, power, and the like output from the control device 9 to the endoscope camera head 5. Here, the setting data is a control signal, a synchronization signal, a clock signal, and the like for controlling the endoscope camera head 5.

Under the control of the control device 9, the display device 7 displays an observation image based on an imaging signal subjected to image processing in the control device 9 and various types of information regarding the endoscope system 1. The display device 7 is realized by using a display monitor such as liquid crystal or organic electro luminescence (EL).

One end of the second transmission cable 8 is detachably connected to the display device 7, and the other end thereof is detachably connected to the control device 9. The second transmission cable 8 transmits the imaging signal subjected to the image processing in the control device 9 to the display device 7.

The control device 9 is realized by using a processor which is a processing device having hardware such as a graphics processing unit (GPU), an FPGA, or a CPU, and a memory which is a temporary storage area used by the processor. The control device 9 integrally controls operations of the light source device 3, the endoscope camera head 5, and the display device 7 via each of the first transmission cable 6, the second transmission cable 8, and the third transmission cable 10 according to a program recorded in the memory. In addition, the control device 9 performs various types of image processing on the imaging signal input via the first transmission cable 6 and outputs the imaging signal to the second transmission cable 8.

One end of the third transmission cable 10 is detachably connected to the light source device 3, and the other end thereof is detachably connected to the control device 9. The third transmission cable 10 transmits the control data from the control device 9 to the light source device 3.

### Functional Configuration of Treatment System

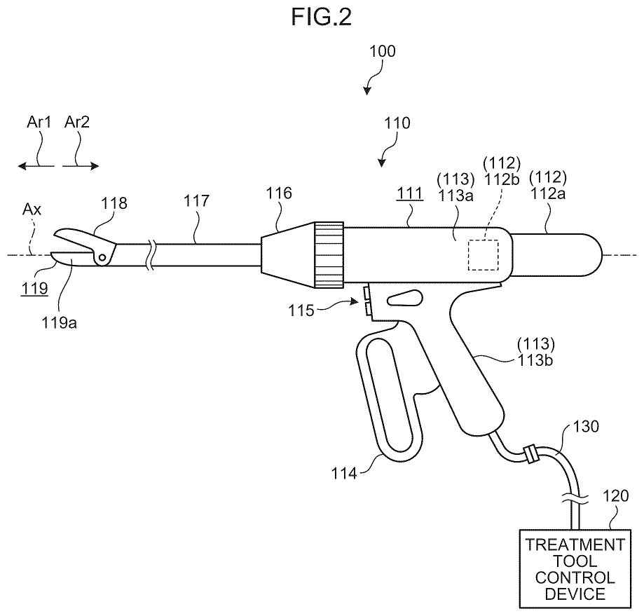

Next, a configuration of a treatment system 100 connected to the above-described endoscope system 1 will be described. FIG. 2 is a diagram illustrating a schematic configuration of a treatment system connected to the endoscope system according to the first embodiment. In FIG. 2, one side along a central axis Ax of the treatment tool is referred to as a distal end side Ar1, and the other side is referred to as a proximal end side Ar2.

The treatment system 100 applies ultrasound energy and high frequency energy to a site to be treated in a biological tissue (hereinafter referred to as a target site), thereby treating the target site. Note that the treatment that can be performed by the treatment system according to the present embodiment is a treatment of coagulating and sealing a target site, a treatment of incising the target site, a treatment of simultaneously performing coagulation and incision, or the like. Then, the treatment system 100 includes a treatment tool 110 and a treatment tool control device 120.

The treatment tool 110 is an ultrasound treatment tool that treats a target site by applying ultrasound energy and high frequency energy to the target site, and corresponds to a surgical device. The treatment tool 110 includes a handpiece 111 and an ultrasound transducer 112. The handpiece 111 includes a holding case 113, a movable handle 114, a switch 115, a rotary knob 116, a pipe 117, a jaw 118, and a vibration transmission member 119.

The ultrasound transducer 112 includes a TD (transducer) case 112a and an ultrasound transducer 112b.

The TD case 112a supports the ultrasound transducer 112b and is detachably connected to a holding case main body 113a.

The ultrasound transducer 112b generates ultrasound vibration under the control of the treatment tool control device 120. In the present embodiment, the ultrasound transducer 112b is configured by a BLT (bolted Langevin transducer).

The holding case 113 constitutes the appearance of the treatment tool 110 and supports the entire treatment tool 110. The holding case 113 includes the substantially cylindrical holding case main body 113a coaxial with the central axis Ax, and a fixed handle 113b formed to extend downwards in FIG. 2 from the main body of the holding case 113 and gripped by an operating person such as an operator.

The movable handle 114 receives an opening/closing operation by an operating person such as an operator. The opening/closing operation is an operation of opening and closing the jaw 118 with respect to an end portion 119a on the distal end side Ar1 of the vibration transmission member 119.

The switch 115 is provided in a state of being exposed to the outside from the side surface of the distal end side Ar1 in the fixed handle 113b. Then, the switch 115 receives a treatment operation by an operating person such as an operator. The treatment operation is an operation of applying ultrasound energy or high frequency energy to a target site. In the present embodiment, the switch 115 is provided with a plurality of buttons, and an operation instruction is assigned to each of the buttons. For example, there are a button for performing treatment as an incision mode and a button for performing treatment as a sealing mode.

The rotary knob 116 has a substantially cylindrical shape coaxial with the central axis Ax, and is provided on the distal end side Ar1 of the holding case main body 113a. Then, the rotary knob 116 receives a rotation operation by an operating person such as an operator. By the rotation operation, the rotary knob 116 is rotated around the central axis Ax with respect to the holding case main body 113a. The rotation of the rotary knob 116 rotates the pipe 117, the jaw 118, and the vibration transmission member 119 around the central axis Ax.

The pipe 117 is a cylindrical pipe. A pin (not illustrated) for rotatably and axially supporting the jaw 118 is fixed to an end portion of the distal end side Ar1 of the pipe 117.

At least a part of the jaw 118 is made of a conductive material. Then, the jaw 118 is opened and closed with respect to the end portion 119a on the distal end side Ar1 of the vibration transmission member 119 in response to a gripping operation on the movable handle 114 by an operating person such as an operator, and grips a target site with the end portion 119a.

The vibration transmission member 119 is made of a conductive material and has an elongated shape extending linearly along the central axis Ax. The vibration transmission member 119 is inserted into the pipe 117 in a state in which the end portion 119a on the distal end side Ar1 protrudes to the outside. At this time, although not specifically illustrated, an end portion of the vibration transmission member 119 on the proximal end side Ar2 is mechanically connected to the ultrasound transducer 112. That is, the vibration transmission member 119 transmits the ultrasound vibration generated by the ultrasound transducer 112 from the end portion on the proximal end side Ar2 to the end portion 119a on the distal end side Ar1. In the present embodiment, the ultrasound vibration is longitudinal vibration that vibrates in a direction along the central axis Ax.

The treatment tool control device 120 comprehensively controls the operation of the treatment tool 110 via an electric cable 130. The treatment tool control device 120 corresponds to a device control device.

Specifically, the treatment tool control device 120 detects a treatment operation on the switch 115 by an operating person such as an operator via the electric cable 130. Then, when detecting the treatment operation, the treatment tool control device 120 applies ultrasound energy or high frequency energy to the target site gripped between the jaw 11 and the end portion 119a on the distal end side Ar1 of the vibration transmission member 119 via the electric cable 130. That is, the treatment tool control device 120 treats the target site.

For example, when applying ultrasound energy to a target site, the treatment tool control device 120 supplies drive power to the ultrasound transducer 112b via the electric cable 130. As a result, the ultrasound transducer 112b generates longitudinal vibration (ultrasound vibration) that vibrates in a direction along the central axis Ax. In addition, the end portion 119a on the distal end side Ar1 of the vibration transmission member 119 vibrates with a desired amplitude by the longitudinal vibration. Then, ultrasound vibration is applied from the end portion 119a to the target site gripped between the jaw 118 and the end portion 119a. In other words, ultrasound energy is applied to the target site from the end portion 119a.

In addition, for example, when applying high-frequency energy to a target site, the treatment tool control device 120 supplies high-frequency power between the jaw 118 and the vibration transmission member 119 via the electric cable 130. As a result, a high-frequency current flows through the target site gripped between the jaw 118 and the end portion 119a on the distal end side Ar1 of the vibration transmission member 119. In other words, high-frequency energy is applied to the target site.

In addition, the treatment tool control device 120 is communicably connected to the control device 9, and outputs a signal indicating pressing of the switch to the control device 9 when the switch 115 is pressed. The treatment tool control device 120 outputs information regarding the output mode of the treatment tool 110 to the control device 9.

### Functional Configuration of Main Part of Endoscope System

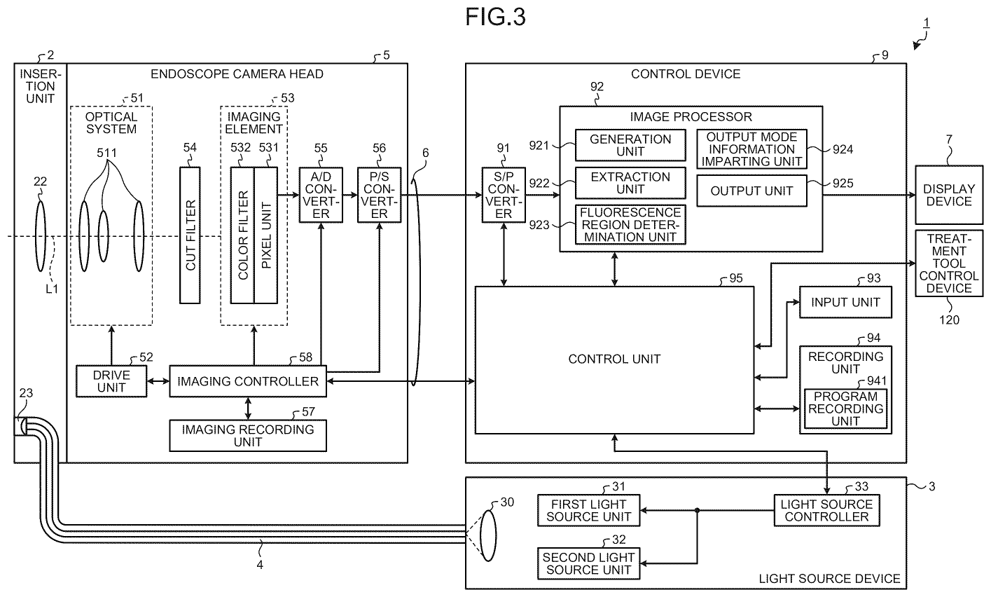

Next, a functional configuration of a main part of the above-described endoscope system 1 will be described. FIG. 3 is a block diagram illustrating a functional configuration of a main part of the endoscope system 1.

### Configuration of Insertion Unit

First, the configuration of the insertion unit 2 will be described. The insertion unit 2 includes an optical system 22 and an illumination optical system 23.

The optical system 22 forms a subject image by collecting light such as reflected light reflected from the subject, return light from the subject, excitation light from the subject, and emission light emitted by the subject. The optical system 22 is realized by using one or a plurality of lenses and the like.

The illumination optical system 23 irradiates the subject with illumination light supplied from the light guide 4. The illumination optical system 23 is realized by using one or a plurality of lenses or the like.

### Configuration of Light Source Device

Next, a configuration of the light source device 3 will be described. The light source device 3 includes a condenser lens 30, a first light source unit 31, a second light source unit 32, and a light source controller 33.

The condenser lens 30 condenses light emitted by each of the first light source unit 31 and the second light source unit 32 and emits the light to the light guide 4.

Under the control of the light source controller 33, the first light source unit 31 supplies white light as illumination light to the light guide 4 by emitting white light (normal light) which is visible light. The first light source unit 31 includes a collimator lens, a white LED lamp, a drive driver, and the like. Note that the first light source unit 31 may supply visible white light by simultaneously emitting light using a red LED lamp, a green LED lamp, and a blue LED lamp. Of course, the first light source unit 31 may be configured using a halogen lamp, a xenon lamp, or the like.

Under the control of the light source controller 33, the second light source unit 32 emits narrow band light in a wavelength band different from white light and a wavelength band narrower than this wavelength band, thereby supplying the narrow band light to the light guide 4 as illumination light. Here, the narrow band light is, for example, light in a wavelength band ranging from 400 nm to 430 nm with a center wavelength of 415 nm. The second light source unit 32 is realized by using a semiconductor laser such as a collimator lens or a violet laser diode (LD), a drive driver, and the like. In the embodiment, the narrow band light functions as excitation light that excites advanced glycation end-products generated by subjecting a biological tissue to thermal treatment.

The light source controller 33 is realized by using a processor which is a processing device having hardware such as an FPGA or a CPU, and a memory which is a temporary storage area used by the processor. The light source controller 33 controls light emission timing, light emission time, and the like of each of the first light source unit 31 and the second light source unit 32 based on control data input from the control device 9.

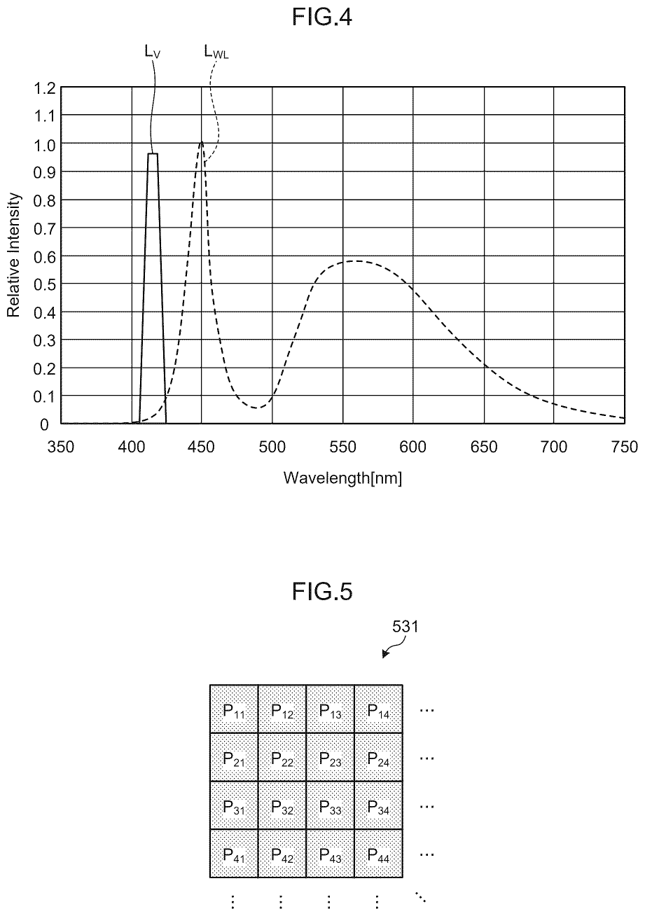

Here, wavelength characteristics of light emitted by the first light source unit 31 and the second light source unit 32 will be described. FIG. 4 is a diagram schematically illustrating wavelength characteristics of light emitted by each of the first light source unit 31 and the second light source unit 32. In FIG. 4, the horizontal axis represents a wavelength (nm), and the vertical axis represents a relative intensity. In FIG. 4, a curve L_{WL} indicates a wavelength characteristic of white light emitted by the first light source unit 31, and a curve L_{V} indicates a wavelength characteristic of narrow band light (excitation light) emitted by the second light source unit 32. The second light source unit 32 has a center wavelength (peak wavelength) of 415 nm and emits light including a wavelength band ranging from 400 nm to 430 nm. The wavelength characteristic indicated by the curve L_{WL} in FIG. 4 indicates a characteristic when the white LED is adopted as the first light source unit 31.

### Configuration of Endoscope Camera Head

Referring back to FIG. 3, the description of the configuration of the endoscope system 1 will be continued.

Next, a configuration of the endoscope camera head 5 will be described. The endoscope camera head 5 includes an optical system 51, a drive unit 52, an imaging element 53, a cut filter 54, an A/D converter 55, a P/S converter 56, an imaging recording unit 57, and an imaging controller 58.

The optical system 51 forms a subject image collected by the optical system 22 of the insertion unit 2 on the light receiving surface of the imaging element 53. The optical system 51 can change the focal length and the focal position. The optical system 51 includes a plurality of lenses 511. The optical system 51 changes the focal length and the focal position by moving each of the plurality of lenses 511 on an optical axis L1 by the drive unit 52.

Under the control of the imaging controller 58, the drive unit 52 moves the plurality of lenses 511 of the optical system 51 along the optical axis L1. The drive unit 52 includes motors such as a stepping motor, a DC motor, and a voice coil motor, and a transmission mechanism such as a gear that transmits rotation of the motor to the optical system 51.

The imaging element 53 is implemented by using a charge coupled device (CCD) or complementary metal oxide semiconductor (CMOS) image sensor having a plurality of pixels arranged in a two-dimensional matrix. Under the control of the imaging controller 58, the imaging element 53 receives a subject image (light beam) that is formed by the optical system 51 and passes through the cut filter 54, performs photoelectric conversion, generates an imaging signal (PAW data), and outputs the imaging signal to the A/D converter 55. The imaging element 53 includes a pixel unit 531 and a color filter 532.

FIG. 5 is a diagram schematically illustrating a configuration of the pixel unit 531. In the pixel unit 531, a plurality of pixels Pₙₘ (n and m are integers of 1 or more) such as photodiodes that accumulate charges according to the amount of light are arranged in a two-dimensional matrix. Under the control of the imaging controller 58, the pixel unit 531 reads an image signal as image data from a pixel Pₙₘ in a reading region arbitrarily set as a reading target among the plurality of pixels Pₙₘ, and outputs the image signal to the A/D converter 55.

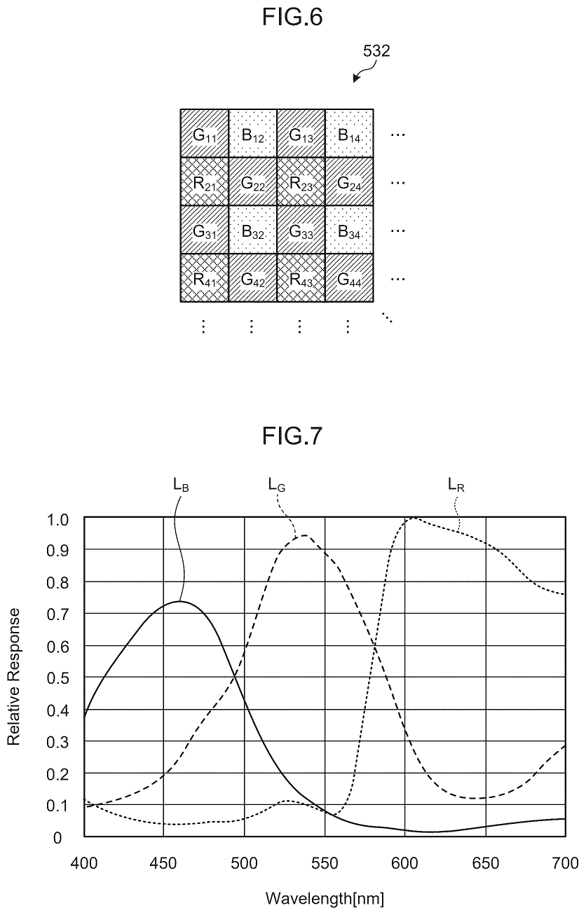

FIG. 6 is a diagram schematically illustrating a configuration of the color filter 532. The color filter 532 is configured by a Bayer array having 2×2 as one unit. The color filter 532 includes a filter R that transmits light in a red wavelength band, two filters G that transmit light in a green wavelength band, and a filter B that transmits light in a blue wavelength band. Note that, in FIG. 5, a reference sign (for example, G₁₁) attached to each filter corresponds to the pixel Pₙₘ and indicates that the filter is arranged at the corresponding pixel position.

FIG. 7 is a diagram schematically illustrating sensitivity characteristics of each filter. In FIG. 7, the horizontal axis represents a wavelength (nm), and the vertical axis represents transmission characteristics (sensitivity characteristics). In FIG. 7, a curve L_{B} represents the transmission characteristics of the filter B, a curve L_{G} represents the transmission characteristic of the filter G, and a curve L_{R} represents the transmission characteristic of the filter R.

The filter B transmits light in a blue wavelength band (refer to the curve L_{B} in FIG. 7). In addition, the filter G transmits light in a green wavelength band (refer to the curve L_{G} in FIG. 7). In addition, the filter R transmits light in a red wavelength band (refer to the curve L_{R} in FIG. 7). Note that, in the following description, a pixel Pₙₘ in which the filter R is arranged on the light receiving surface will be described as an R pixel, a pixel Pₙₘ in which the filter G is arranged on the light receiving surface will be described as a G pixel, and a pixel Pₙₘ in which the filter B is arranged on the light receiving surface will be described as a B pixel.

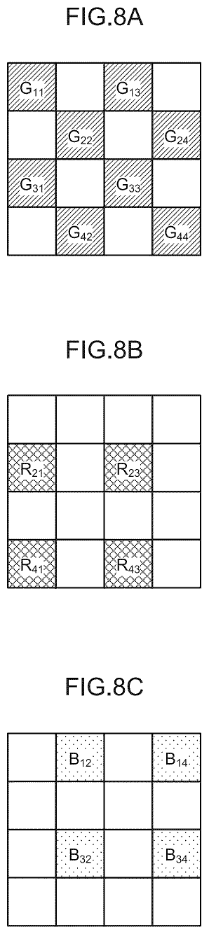

According to the imaging element 53 configured as described above, in a case where the subject image formed by the optical system 51 is received, a color signal (R signal, G signal, and B signal) of each of the R pixel, the G pixel, and the B pixel is generated (refer to FIGS. 8A to 8C).

Referring back to FIG. 3, the description of the configuration of the endoscope system 1 will be continued. The cut filter 54 is disposed on the optical axis L1 between the optical system 51 and the imaging element 53. The cut filter 54 is provided on the light receiving surface side (incident surface side) of the G pixel provided with the filter G that transmits at least the green wavelength band of the color filter 532. The cut filter 54 shields light in a wavelength band of excitation light and transmits a wavelength band longer than the wavelength band of the excitation light.

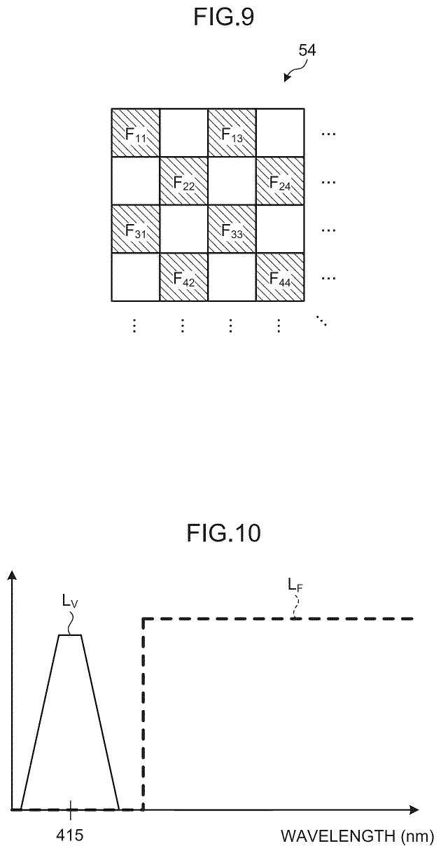

FIG. 9 is a diagram schematically illustrating a configuration of the cut filter 54. As illustrated in FIG. 9, a filter F₁₁ constituting the cut filter 54 is arranged at a position where the filter G₁₁ (refer to FIG. 6) is arranged, and is arranged on the light receiving surface side directly above the filter G₁₁.

FIG. 10 is a diagram schematically illustrating transmission characteristics of the cut filter 54. In FIG. 10, the horizontal axis represents a wavelength (nm), and the vertical axis represents transmission characteristics. In FIG. 10, a curve L_{F} indicates the transmission characteristics of the cut filter 54, and a curve L_{V} indicates the wavelength characteristics of excitation light.

The cut filter 54 shields the wavelength band of the excitation light and transmits the wavelength band on the long wavelength side from the wavelength band of the excitation light. Specifically, the cut filter 54 shields light in a wavelength band equal to or less than the wavelength band of excitation light and transmits light in a wavelength band longer than the excitation light.

Referring back to FIG. 3, the description of the configuration of the endoscope camera head 5 will be continued.

Under the control of the imaging controller 58, the A/D converter 55 performs A/D conversion processing on an analog imaging signal input from the imaging element 53, and outputs the analog imaging signal to the P/S converter 56. The A/D converter 55 is implemented by using an A/D conversion circuit or the like.

Under the control of the imaging controller 58, the P/S converter 56 performs parallel/serial conversion on a digital imaging signal input from the A/D converter 55, and outputs the imaging signal subjected to the parallel/serial conversion to the control device 9 via the first transmission cable 6. The P/S converter 56 is implemented by using a P/S conversion circuit or the like. Note that, in the first embodiment, an E/O converter that converts an imaging signal into an optical signal may be provided instead of the P/S converter 56, and the imaging signal may be output to the control device 9 by the optical signal, or the imaging signal may be transmitted to the control device 9 by, for example, wireless communication such as Wireless Fidelity (Wi-Fi) (registered trademark.

The imaging recording unit 57 records various types of information (for example, pixel information of the imaging element 53 and characteristics of the cut filter 54) regarding the endoscope camera head 5. Furthermore, the imaging recording unit 57 records various setting data and control parameters transmitted from the control device 9 via the first transmission cable 6. The imaging recording unit 57 is configured using a nonvolatile memory or a volatile memory.

The imaging controller 58 controls the operation of each of the drive unit 52, the imaging element 53, the A/D converter 55, and the P/S converter 56 based on the setting data received from the control device 9 via the first transmission cable 6. The imaging controller 58 is implemented by using a timing generator (TG), a processor which is a processing device having hardware such as a CPU, and a memory which is a temporary storage area used by the processor.

### Configuration of Control Device

Next, a configuration of the control device 9 will be described.

The control device 9 includes an S/P converter 91, an image processor 92, an input unit 93, a recording unit 94, and a control unit 95.

Under the control of the control unit 95, the S/P converter 91 performs serial/parallel conversion on image data received from the endoscope camera head 5 via the first transmission cable 6 and outputs the image data to the image processor 92. Note that, in a case where the endoscope camera head 5 outputs an imaging signal as an optical signal, an O/E converter that converts an optical signal into an electric signal may be provided instead of the S/P converter 91. Furthermore, in a case where the endoscope camera head 5 transmits an imaging signal by wireless communication, a communication module capable of receiving a wireless signal may be provided instead of the S/P converter 91.

Under the control of the control unit 95, the image processor 92 performs predetermined image processing on an imaging signal of parallel data input from the S/P converter 91 and outputs the imaging signal to the display device 7. Here, the predetermined image processing is demosaic processing, white balance processing, gain adjustment processing, y correction processing, format conversion processing, and the like. The image processor 92 is implemented by using a processor which is a processing device having hardware such as a GPU or an FPGA and a memory which is a temporary storage area used by the processor. The image processor 92 includes a generation unit 921, an extraction unit 922, a fluorescence region determination unit 923, an output mode information imparting unit 924, and an output unit 925.

The generation unit 921 generates a first image including one or more characteristic regions that need to be resected by an operator and a second image including one or more cauterized regions cauterized by the energy device (the treatment tool 110). Specifically, the generation unit 921 generates a white light image, which is a first image, based on an imaging signal generated by capturing reflected light when the biological tissue is irradiated with white light and return light from the biological tissue. In addition, in a fluorescence observation mode to be described later, the generation unit 921 generates a fluorescence image, which is a second image, based on an imaging signal generated by capturing fluorescence generated by excitation light emitted for exciting advanced glycation end-products generated by applying thermal treatment to a biological tissue. Here, the generation unit 921 may generate a pseudo color image, which is a pseudo color image including one or more characteristic regions (lesion regions) that need to be resected by the operator, based on an imaging signal obtained by imaging reflected light when excitation light is emitted to a biological tissue and return light from the biological tissue in a fluorescence observation mode of the endoscope system 1 to be described later.

The extraction unit 922 extracts a fluorescence region, which is a region of a fluorescence shape, from the fluorescence image generated by the generation unit 921.

The fluorescence region determination unit 923 determines the presence or absence of a change in the fluorescence region between fluorescence images having different imaging times.

The output mode information imparting unit 924 imparts output mode information to a fluorescence region (a fluorescence shape) based on a determination result of an output mode. Specifically, information on the output mode when the fluorescent region is generated is imparted to the fluorescence region. Examples of the output mode include an incision mode in which a biological tissue is incised and a sealing (coagulation) mode in which an incision portion is sealed.

The output unit 925 outputs a white light image, a fluorescence image, and a fluorescence image to which the output mode information is imparted by the output mode information imparting unit 924. In addition, the output unit 925 outputs a white light image to which the output mode information is imparted by the output mode information imparting unit 924.

The input unit 93 receives inputs of various operations related to the endoscope system 1 and outputs the received operations to the control unit 95. The input unit 93 includes a mouse, a foot switch, a keyboard, a button, a switch, a touch panel, and the like.

The recording unit 94 is implemented by using a recording medium such as a volatile memory, a nonvolatile memory, a solid state drive (SSD), a hard disk drive (HDD), or a memory card. The recording unit 94 records data including various parameters and the like necessary for the operation of the endoscope system 1. Furthermore, the recording unit 94 includes a program recording unit 941 that records various programs for operating the endoscope system 1.

The control unit 95 is implemented by using a processor which is a processing device having hardware such as an FPGA or a CPU, and a memory which is a temporary storage area used by the processor. The control unit 95 integrally controls each of the units constituting the endoscope system 1. In addition, the control unit 95 receives a signal related to pressing of the switch 115 (output of the treatment tool 110) from the treatment tool control device 120.

### Outline of Each Observation Mode

Next, an outline of each observation mode that can be executed by the endoscope system 1 will be described. In the following description, the normal light observation mode and the fluorescence observation mode will be described in this order.

### Outline of Normal Light Observation Mode

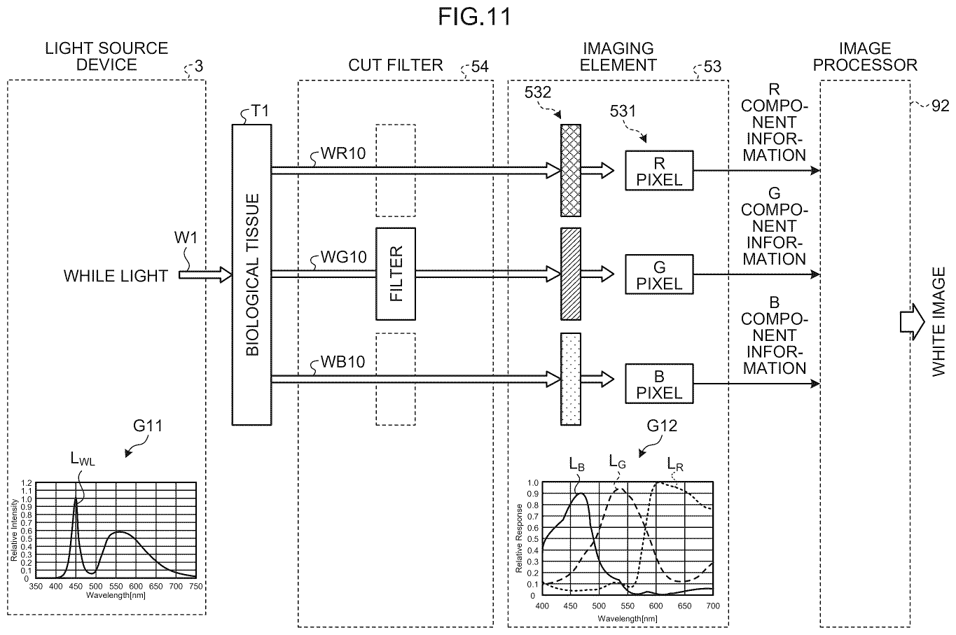

First, the normal light observation mode will be described. FIG. 11 is a diagram schematically illustrating an observation principle in the normal light observation mode.

Under the control of the control device 9, the light source device 3 irradiates a biological tissue T1 of a subject with a white light W1 having an intensity distribution illustrated in a graph G11 by causing the first light source unit 31 to emit light. In this case, a part of the reflected light and the return light (hereinafter, simply referred to as “reflected light WR10, reflected light WG10, and reflected light WB10”) reflected by the biological tissue is shielded by the cut filter 54, and the rest of the light is incident on the imaging element 53. For example, specifically, the cut filter 54 shields reflected light (the reflected light WG10) which is incident on the G pixel and is in a wavelength band of excitation light (excitation light W2 to be described later). That is, reflected light and return light based on irradiation of white light are incident on the filter R and the filter B, and light in a wavelength band longer than the wavelength band of the excitation light is incident on the filter G. Therefore, a component of light in a blue wavelength band incident on the pixel is smaller than that in a state in which the cut filter 54 is not disposed. The light incident on each filter is selectively transmitted by filter characteristics illustrated in the graph G12.

Subsequently, the image processor 92 acquires image data (RAW data) from the imaging element 53 of the endoscope camera head 5, and performs image processing on signal values of the R pixel, the G pixel, and the B pixel included in the acquired image data to generate a white light image. In this case, since a blue component included in the image data is smaller than that in the conventional white light observation, the image processor 92 performs white balance adjustment processing of adjusting white balance so that a ratio of a red component, a green component, and the blue component is constant.

In the normal light observation mode, even in a case where the cut filter 54 is arranged on the light receiving surface side of the G pixel, a natural white light image (observation image) can be observed.

### Outline of Fluorescence Observation Mode

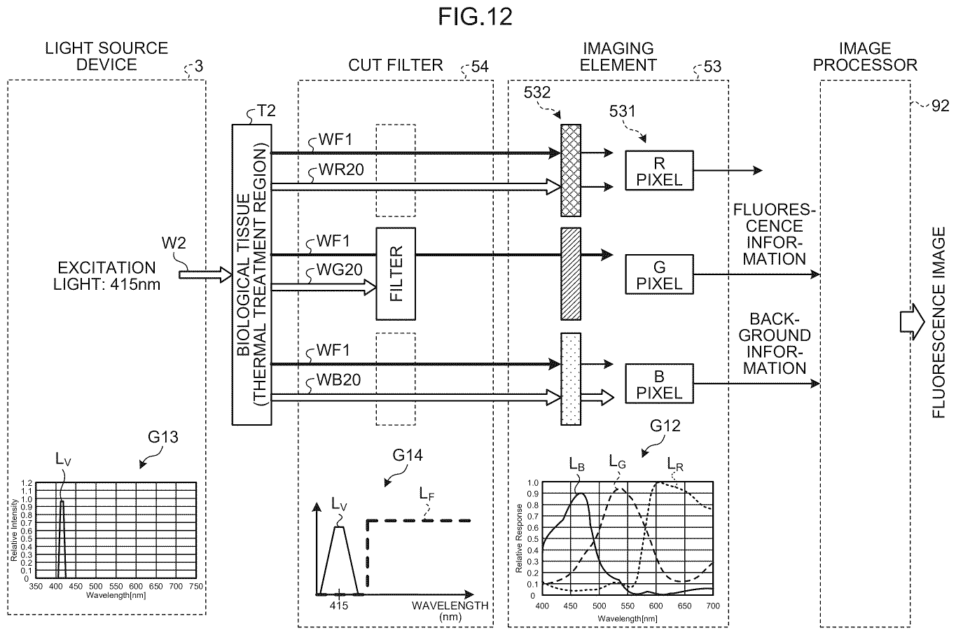

Next, the fluorescence observation mode will be described. FIG. 12 is a view schematically illustrating an observation principle in the fluorescence observation mode.

In recent years, in the medical field, minimally invasive treatment using an endoscope, a laparoscope, or the like has been widely performed. For example, as minimally invasive treatment using an endoscope, a laparoscope, or the like, endoscopic submucosal dissection (ESD), laparoscopy and endoscopy cooperative surgery (LECS), non-exposed endoscopic wall-inversion surgery (NEWS), transurethral resection of the bladder tumor (TUR-bt), or the like is widely performed.

In these minimally invasive treatments, in the case of performing treatment, for example, an operator such as a doctor performs thermal treatment using a treatment tool of an energy device that emits energy such as high frequency, ultrasonic waves, and microwaves, and marks a region to be operated as pretreatment, or excises a lesion, seals an incision, or coagulates the sealed incision as treatment.

By the way, when an amino compound and a reducing sugar are heated, a saccharification reaction (Maillard reaction) in which the amino acid and the reducing sugar react with each other occurs. The end product generated by this Maillard reaction is generally called advanced glycation end-products (AGEs). As characteristics of these AGEs, it is known that a substance having fluorescence characteristics is included. AGEs are known to emit fluorescence with higher intensity than autofluorescent substances originally present in biological tissues. Therefore, due to the generation of the AGEs, the fluorescence intensity significantly increases as compared with before the AGEs are generated.

AGEs generated by cauterization at the time of treatment can be visualized by observation of fluorescence, and its fluorescence intensity is an indicator of the state of thermal treatment. The fluorescence at this time corresponds to a trajectory of the distal end (the jaw 118 and the vibration transmission member 119) of the energy device (the treatment tool 110).

That is, the fluorescence observation mode is an observation mode for visualizing a thermal treatment region using the fluorescence characteristics of the AGEs generated in the biological tissue by being subjected to thermal treatment by an energy device or the like. Therefore, in the fluorescence observation mode, the biological tissue is irradiated with excitation light for exciting the AGEs from the light source device 3, for example, blue narrow band light having a center wavelength of 415 nm. As a result, in the fluorescence observation mode, it is possible to observe a thermal treatment image (fluorescence image) obtained by imaging fluorescence (for example, green light having a wavelength ranging from 490 nm to 625 nm) generated from the AGEs.

Specifically, first, the light source device 3 causes the second light source unit 32 to emit light under the control of the control device 9, thereby irradiating a biological tissue T2 (thermal treatment region) subjected to the thermal treatment on the subject by the energy device or the like with excitation light W2 (center wavelength 415 nm: refer to a graph G13). In this case, reflected light (hereinafter, simply referred to as “reflected light WR20, reflected light WG20, and reflected light WB20”) including at least a component of the excitation light W2 and return light reflected by the biological tissue T2 (thermal treatment region) is blocked by the cut filter 54, and a part of the component on the long wavelength side is incident on the imaging element 53 (refer to a graph G14). In FIGS. 11 and 12, the intensity of a component (a light amount or a signal value) of each line is expressed by the thickness of an arrow.

More specifically, as illustrated in a graph G14 of FIG. 12, the cut filter 54 shields the reflected light WG20 incident on the G pixel, which is the reflected light WG20 in the wavelength band including the wavelength band of the excitation light W2. Furthermore, the cut filter 54 transmits fluorescence WF1 self-emitted by the AGEs in the biological tissue T2 (thermal treatment region) (refer to the graph G14). Therefore, the reflected light WG20 is not incident on the G pixel, and the fluorescence WF1 is incident on the G pixel. In the G pixel, since the cut filter 54 is arranged on the light receiving surface side (incident surface side), it is possible to prevent a fluorescence component from being buried due to mixing of the reflected light WG20 of the excitation light W2 with the fluorescence WF1.

Furthermore, the reflected light (the reflected light WR20, WB20) and the fluorescence WF1 are incident on the R pixel and the B pixel, respectively.

Thereafter, the image processor 92 acquires image data (PAW data) from the imaging element 53 of the endoscope camera head 5, and performs image processing on signal values of the G pixel and the B pixel included in the acquired image data to generate a fluorescence image. In this case, the signal value of the G pixel includes fluorescence information indicating a fluorescence shape emitted from the thermal treatment region. Furthermore, the B pixel includes background information which is a biological tissue around a thermal treatment region and forms a background of the thermal treatment region. The image processor 92 performs image processing such as gain control processing, pixel complement processing, and mucosal enhancement processing on the signal value of each of the G pixel and the B pixel included in the image data to generate a fluorescence image. At this time, in the gain control processing, the image processor 92 performs processing of making a gain for the signal value of the G pixel larger than a gain for the signal value of the G pixel at the time of normal light observation, and making a gain for the signal value of the B pixel smaller than a gain for the signal value of the B pixel at the time of normal light observation. Furthermore, the image processor 92 performs processing in which the signal value of the G pixel and the signal value of the B pixel are the same (1:1). Note that the image processor 92 may generate a pseudo color image in which color information in which hue is changed according to fluorescence intensity is superimposed on a fluorescence shape.

### Treatment Using Endoscope System

Next, treatment using the endoscope system 1 of the present disclosure will be described. At this time, the operator inserts the insertion unit 2 into a subject, causes the light source device 3 to irradiate the inside of the subject with white light, and irradiates a region including a treatment target with white light. An operator confirms the treatment target while observing an observation image displayed by the display device 7.

Thereafter, the operator performs treatment on the treatment target of the subject while confirming the white light image displayed on the display device 7. For example, the operator cauterizes and resects the treatment target using an energy device (the treatment tool 110) or the like inserted into a subject via the insertion unit 2.

Thereafter, the operator irradiates the treatment target with the excitation light and observes the fluorescence image displayed by the display device 7. The operator determines whether the treatment (for example, resection) at the treatment position is completed by observing the fluorescence image displayed by the display device 7. If the operator determines that the procedure is complete, the treatment is terminated. Specifically, the operator determines whether the resection of the treatment target has been completed by observing the fluorescence image displayed by the display device 7 and observing a cauterized region resected by performing cauterization with the treatment tool 110. At this time, in a case where it is determined that the resection of the treatment target is not completed, the operator repeats the observation of the white light image by the irradiation of the white light and the observation of the fluorescence image by the irradiation of the excitation light while switching the observation mode of the endoscope system 1, and continues the treatment.

### Processing of Endoscope System

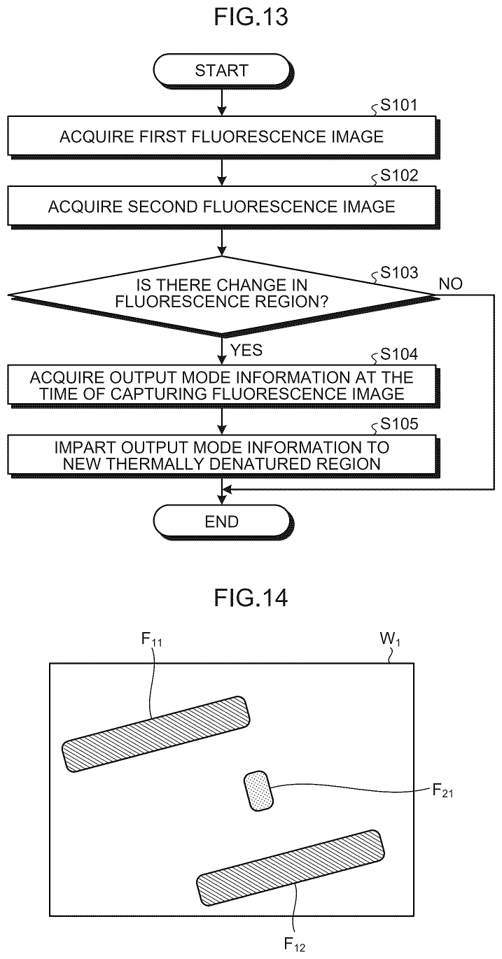

Next, processing executed by the endoscope system 1 will be described. FIG. 13 is a flowchart for description of thermally denatured region determination processing using the endoscope system according to the embodiment. The thermally denatured region determination processing is processing executed in the fluorescence observation mode.

The control unit 95 generates a first fluorescence image (step S101). At this time, the control unit 95 controls the light source controller 33 to cause the second light source unit 32 to emit light, and irradiates the subject with excitation light. The generation unit 921 generates the first fluorescence image by acquiring an imaging signal from the imaging element 53 of the endoscope camera head 5. As a result, the first fluorescence image is acquired. In this case, the output unit 925 may cause the display device 7 to display the first fluorescence image generated by the generation unit 921.

Subsequently, the control unit 95 generates a second fluorescence image (step S102). At this time, the control unit 95 controls the light source controller 33 to cause the second light source unit 32 to emit light, and irradiates the subject with excitation light. The generation unit 921 generates the second fluorescence image by acquiring an imaging signal from the imaging element 53 of the endoscope camera head 5. As a result, the second fluorescence image is acquired. In this case, the output unit 925 may cause the display device 7 to display the second fluorescence image generated by the generation unit 921.

The second fluorescence image is a fluorescence image based on image data acquired at a time later than the first fluorescence image. An acquisition time (imaging timing) of the image data is executed, for example, after a preset time elapses after the first fluorescence image is acquired.

Thereafter, the control unit 95 determines whether there is a change in the fluorescence region between the first fluorescence image and the second fluorescence image (step S103). In this step, the extraction unit 922 extracts a region (fluorescence region) in which the fluorescence shape is drawn from each fluorescence image. For example, the extraction unit 922 extracts one or a plurality of fluorescence regions included in the image by executing contour extraction based on a luminance value or the like. Then, the fluorescence region determination unit 923 determines whether there is a change in the fluorescence region of the second fluorescence image with respect to the fluorescence region of the extracted first fluorescence image. At this time, the fluorescence region determination unit 923 detects the change in the fluorescence region by determining the presence or absence of a new fluorescence region present in the second fluorescence image and not present in the first fluorescence image. When the fluorescence region determination unit 923 determines that there is no change in the fluorescence region (step S103: No), the control unit 95 ends the processing. On the other hand, when the fluorescence region determination unit 923 determines that there is a change in the fluorescence region (step S103: Yes), the control unit 95 proceeds to step S104.

In step S104, the control unit 95 acquires the output mode of the treatment tool 110 when each fluorescence image is captured. Specifically, the control unit 95 acquires information regarding the output mode of the treatment tool 110 from the treatment tool control device 120.

Thereafter, the output mode information imparting unit 924 imparts information regarding the output mode of the treatment tool 110 to the fluorescence region of each fluorescence image according to the time when the first and second fluorescence images are captured. At this time, the output mode information imparting unit 924 may impart the information regarding the output mode to a region exceeding a specific fluorescence intensity.

Here, the temporal change of the fluorescence image will be described with reference to FIG. 14. FIG. 14 is a diagram for description of a fluorescence shape in the fluorescence observation mode. FIG. 14 illustrates an example in which a part is a fluorescence image having a plurality of fluorescence shapes generated by different output modes. Specifically, fluorescence shapes F₁₁ and F₁₂ are generated in the same output mode, and a fluorescence shape F₂₁ is generated in an output mode different from the fluorescence shapes F₁₁ and F₁₂. At this time, the same hue is superimposed on the fluorescence shapes F₁₁ and F₁₂, and a hue different from those of the fluorescence shapes F₁₁ and F₁₂ is superimposed on the fluorescence shape F₂₁. At this time, the fluorescence shape F₂₁ is detected as a change in the fluorescence region by the fluorescence region determination unit 923, and information of an output mode different from those of the fluorescence shapes F₁₁ and F₁₂ is imparted. The generation unit 921 selects a preset hue according to the output mode and superimposes the hue on each fluorescence shape to generate a fluorescence image for display.

In addition to the hue, textual information indicating the output mode may be superimposed. In addition, a fluorescence image of only a fluorescence shape in a designated output mode may be displayed. Furthermore, regions corresponding to the fluorescence shapes F₁₁ and F₁₂ in the fluorescence image may be superimposed on and displayed with a preset hue on the white light image.

The thermally denatured region determination processing is executed, for example, at a preset time interval or at a timing when an execution instruction of detection processing is input from the operator or the like. At this time, the second fluorescence image acquired in the previous processing can be set as the first fluorescence image, and in this case, the processing can be started from step S102.

In the first embodiment described above, when there is a change in the fluorescence region in the fluorescence image, the output mode information of the treatment tool is acquired, and information corresponding to the output mode is imparted to and displayed on the fluorescence shape in the fluorescence image, thereby allowing the operator to grasp the output mode of each fluorescence shape. According to the first embodiment, it is possible to cause the operator to grasp the treatment type of the fluorescence shape in the fluorescence image.

### Second Embodiment

Next, a second embodiment will be described. In the first embodiment described above, the endoscope system includes the rigid endoscope, but in the second embodiment, an endoscope system including a flexible endoscope will be described. Hereinafter, the endoscope system according to the second embodiment will be described. Note that, in the second embodiment, the same components as those of the endoscope system 1 according to the first embodiment described above are denoted by the same reference numerals, and a detailed description thereof will be omitted.

### Configuration of Endoscope System

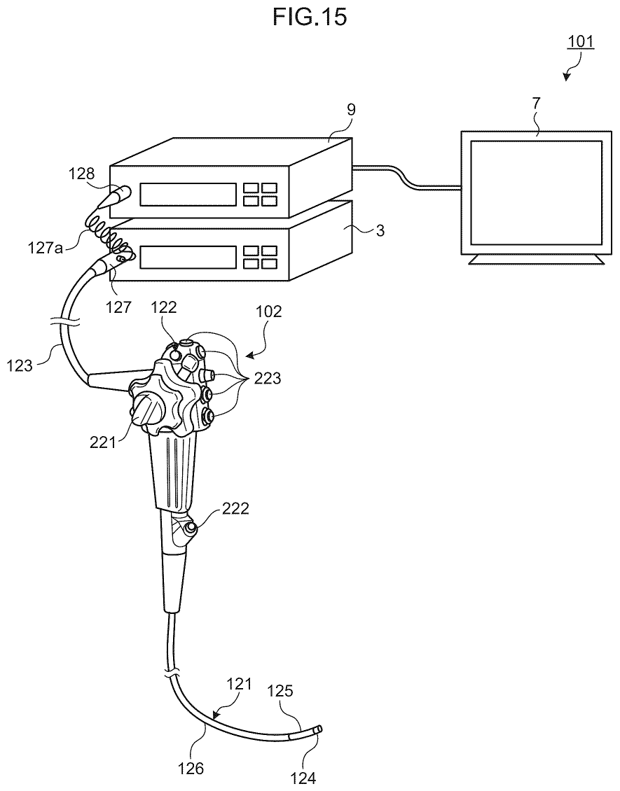

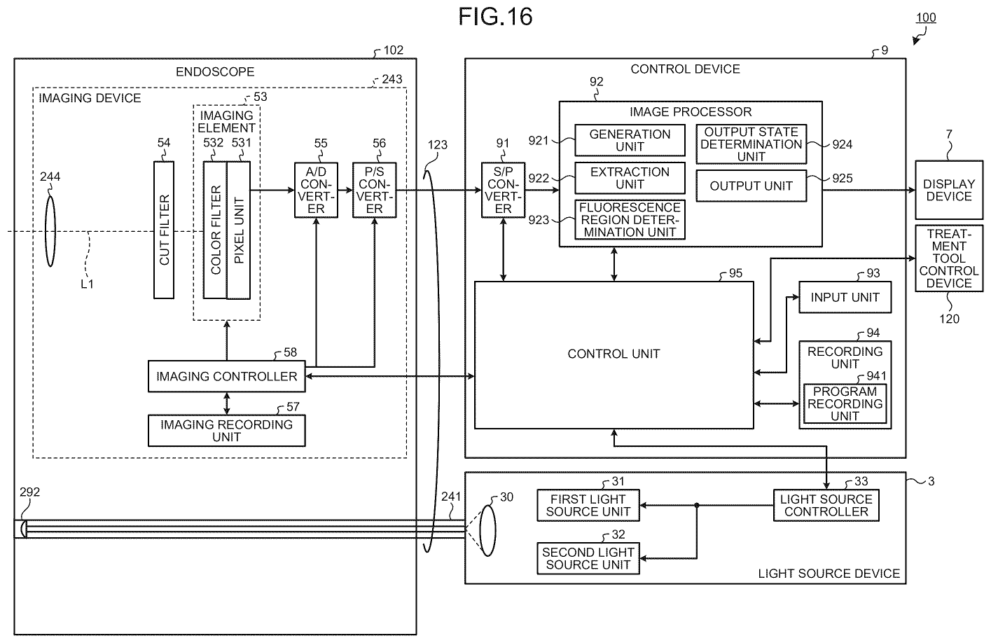

FIG. 15 is a diagram illustrating a schematic configuration of the endoscope system according to the second embodiment. FIG. 16 is a block diagram illustrating a functional configuration of a main part of the endoscope system according to the second embodiment.

An endoscope system 101 is inserted into a subject such as a patient to capture an image of the inside of the subject, and the display device 7 displays a display image based on the captured image data. An operator such as a doctor observes the display image displayed by the display device 7 to examine the presence or absence and the state of each of the bleeding site, the tumor site, and the abnormal region in which the abnormal site appears as the examination target site. Furthermore, an operator such as a doctor inserts a treatment tool such as an energy device into a body of a subject via a treatment tool channel of an endoscope to treat the subject. The endoscope system 101 includes an endoscope 102 in addition to the light source device 3, the display device 7, and the control device 9 described above.

### Configuration of Endoscope

A configuration of the endoscope 102 will be described. The endoscope 102 generates image data by capturing the inside of the body of the subject, and outputs the generated image data to the control device 9. The endoscope 102 includes an operating unit 122 and a universal cord 123.

An insertion unit 121 has an elongated shape having flexibility. The insertion unit 121 includes a distal end portion 124 incorporating an imaging device to be described later, a bendable bending portion 125 including a plurality of bending pieces, and an elongated flexible tube portion 126 connected to a proximal end side of the bending portion 125 and having flexibility.

The distal end portion 124 is configured using glass fiber or the like. The distal end portion 124 includes a light guide 241 forming a light guide path of light supplied from the light source device 3, an illumination lens 242 provided at the distal end of the light guide 241, and an imaging device 243.

The imaging device 243 includes an optical system 244 for condensing light, and the above-described imaging element 53, cut filter 54, A/D converter 55, P/S converter 56, imaging recording unit 57, and imaging controller 58 of the first embodiment.

The universal cord 123 incorporates at least the light guide 241 and a cable assembly including one or a plurality of cables. The assembly cable is a signal line for transmitting and receiving a signal between the endoscope 102 and the light source device 3 and the control device 9, and includes a signal line for transmitting and receiving setting data, a signal line for transmitting and receiving a captured image (image data), a signal line for transmitting and receiving a driving timing signal for driving the imaging element 53, and the like. The universal cord 123 has a connector portion 127 detachable from the light source device 3. The connector portion 127 has a coil-shaped coil cable 127a extending, and a connector portion 128 detachably attached to the control device 9 at an extending end of the coil cable 127a.

The endoscope system 101 configured as described above performs processing similar to that of the endoscope system 1 according to the first embodiment described above.

In the second embodiment described above, similarly to the first embodiment described above, when a change occurs in the fluorescence region in the fluorescence image, the output mode information of the treatment tool is acquired, and information corresponding to the output mode is imparted to and displayed on the fluorescence image in the fluorescence image, thereby allowing the operator to grasp the output mode of each fluorescence shape. According to the second embodiment, it is possible to cause the operator to grasp the treatment type of the fluorescence shape in the fluorescence image.

### Third Embodiment

Next, a third embodiment will be described. In the first and second embodiments described above, the endoscope system is used, but in the third embodiment, a case in which the endoscope system is applied to a surgical microscope system will be described. Note that, in the third embodiment, the same components as those of the endoscope system 1 according to the first embodiment described above are denoted by the same reference numerals, and a detailed description thereof will be omitted.

### Configuration of Surgical Microscope System

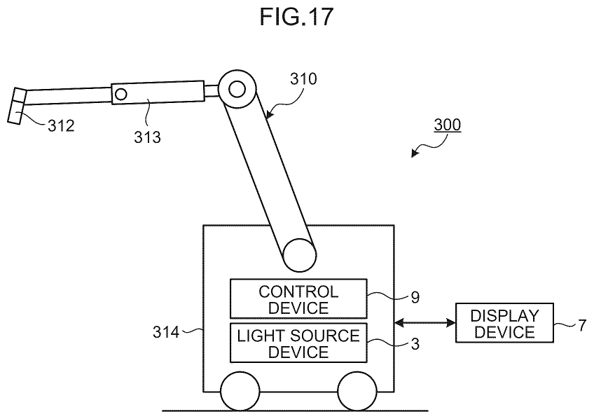

FIG. 17 is a diagram illustrating a schematic configuration of a surgical microscope system according to the third embodiment. A surgical microscope system 300 includes a microscope device 310 which is a medical imaging device that captures and acquires an image for observing a subject, and a display device 7. Note that the display device 7 and the microscope device 310 can also be integrally configured.

The microscope device 310 includes a microscope unit 312 that enlarges and captures a minute portion of a subject, a support unit 313 that is connected to a proximal end portion of the microscope unit 312 and includes an arm that rotatably supports the microscope unit 312, and a base unit 314 that rotatably holds the proximal end portion of the support unit 313 and is movable on a floor surface. The base unit 314 includes a light source device 3 that generates white light, first narrow band light, second narrow band light, and the like to be emitted from the microscope device 310 to the subject, and a control device 9 that controls the operation of the surgical microscope system 300. Note that each of the light source device 3 and the control device 9 has at least a configuration similar to that of the first embodiment described above. Specifically, the light source device 3 includes the condenser lens 30, the first light source unit 31, the second light source unit 32, and the light source controller 33. Furthermore, the control device 9 includes the S/P converter 91, the image processor 92, the input unit 93, the recording unit 94, and the control unit 95. The base unit 314 may be fixed to a ceiling, a wall surface, or the like to support the support unit 313 instead of being movably provided on the floor surface.

The microscope unit 312 has, for example, a cylindrical shape and includes the above-described medical imaging device inside thereof. Specifically, the medical imaging device has a configuration similar to that of the endoscope camera head 5 according to the first embodiment described above. For example, the microscope unit 312 includes the optical system 51, the drive unit 52, the imaging element 53, the cut filter 54, the A/D converter 55, the P/S converter 56, the imaging recording unit 57, and the imaging controller 58. In addition, a switch that receives an input of an operation instruction of the microscope device 310 is provided on the side surface of the microscope unit 312. A cover glass for protecting the inside is provided on the aperture surface of a lower end portion of the microscope unit 312 (not illustrated).

In the surgical microscope system 300 configured as described above, a user such as an operator moves the microscope unit 312, performs a zoom operation, or switches illumination light while operating various switches in a state of holding the microscope unit 312. Note that the shape of the microscope unit 312 is preferably a shape elongated in the observation direction so that the user can easily hold and change the viewing direction. Therefore, the shape of the microscope unit 312 may be a shape other than the columnar shape, and may be, for example, a polygonal columnar shape.

In the third embodiment described above, in the surgical microscope system 300 as well, when is a change in the fluorescence region in the fluorescence image, the output mode information of the treatment tool is acquired, and information corresponding to the output mode is imparted to and displayed on the fluorescence shape in the fluorescence image, thereby allowing the operator to grasp the output mode of each fluorescence shape, similarly to the first embodiment described above. According to the third embodiment, it is possible to cause the operator to grasp the treatment type of the fluorescence shape in the fluorescence image.

### OTHER EMBODIMENTS

Various embodiments can be formed by appropriately combining a plurality of components disclosed in the endoscope system according to the first and second embodiments of the present disclosure or the surgical microscope system according to the third embodiment. For example, some components may be deleted from all the components described in the endoscope system or the surgical microscope system according to the embodiment of the present disclosure described above. Furthermore, the components described in the endoscope system or the surgical microscope system according to the embodiment of the present disclosure described above may be appropriately combined. Furthermore, the present embodiment can be applied as long as the treatment is performed based on fluorescence emitted by a substance generated by cauterization or the like.

Furthermore, in the embodiment and the modification, the processing example has been described on the assumption that the first and second fluorescence images are images having the same angle of view. However, in a case where images having different angles of view and partially including the same subject are used, the fluorescence regions (thermally denatured regions) are associated with each other using a known method such as pattern matching, and change detection of the fluorescence region and setting processing of the thermally denatured region generated after the output is turned off are executed.

Furthermore, in the endoscope system or the surgical microscope system according to the embodiment of the present disclosure, the above-described “unit” can be replaced with “means”, “circuit”, or the like. For example, the control unit can be replaced with a control means or a control circuit.

Furthermore, in the description of the flowcharts in the present specification, the context of processing between steps is clearly indicated using expressions such as “first”, “thereafter”, and “subsequently”, but the order of processing necessary for implementing the embodiments is not uniquely determined by such expressions. That is, the order of processing in the flowcharts described in the present specification can be changed within a range without inconsistency.

In addition, the program to be executed by each device according to the first to third embodiments is provided by being recorded as file data in an installable format or an executable format in a computer-readable recording medium such as a CD-ROM, a flexible disk (FD), a CD-R, a digital versatile disk (DVD), a USB medium, or a flash memory.

The program to be executed by each device according to the first to third embodiments may be stored in a computer connected to a network such as the Internet and may be provided by being downloaded via the network. Furthermore, the program to be executed by the information processing device according to the first to third embodiments may be provided or distributed via a network such as the Internet.

Note that, in the first and second embodiments, an example in which the light source device 3 is separated from the control device 9 has been described, but the light source device 3 and the control device 9 may be configured to be integrated. Furthermore, in the third embodiment, an example in which the light source device 3 is integrated with the control device 9 has been described, but the light source device 3 and the control device 9 may be configured as separate bodies.

Although some of the embodiments of the present application have been described in detail with reference to the drawings, these are merely examples, and the embodiments can be implemented in other forms to which various modifications and improvements have been made based on the knowledge of those skilled in the art, including the aspects described in the section of the present disclosure.

As described above, the medical device, the medical system, the operation method of the medical device, and the operation program of the medical device according to the disclosure are useful for causing an operator to grasp the thermal denaturation generated when the output of the treatment tool is turned off.

According to the present disclosure, there is an effect of enabling an operator to grasp a treatment type of a fluorescence shape (a thermally denatured region) in a fluorescence image.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

### EXAMPLES

[1] A medical device comprising:
   an output mode information imparting unit configured to impart, based on output information of an energy device, information of an output mode of the energy device to a fluorescence region in a fluorescence image based on fluorescence generated by excitation light that excites a substance generated by cauterization using the energy device; and
   an output unit configured to output information in which the information of the output mode is imparted to the fluorescence region by the output mode information imparting unit.
[2] The medical device according to [1], further comprising a generation unit configured to generate the fluorescence image.
[3] The medical device according to [1], wherein
   the output mode is either an incision mode in which a treatment target is incised or a sealing mode in which a treatment target is coagulated and sealed.
[4] The medical device according to [1], further comprising:
   an extraction unit configured to extract fluorescence regions from a first fluorescence image and a second fluorescence image, the fluorescence regions being generated by different output modes of the energy device; and
   a fluorescence region determination unit configured to determine presence or absence of a new fluorescence region present only in the second fluorescence image for the fluorescence regions extracted by the extraction unit, wherein
   the output mode information imparting unit is further configured to impart information of the different output modes to the new fluorescence region and a fluorescence region other than the new fluorescence region.
[5] The medical device according to [4], wherein
   the second fluorescence image is captured later in time than the first fluorescence image.
[6] The medical device according to [1], wherein
   the fluorescence is light generated by excitation of the substance.
[7] The medical device according to [6], wherein
   the substance is an advanced glycation end-product generated by thermal denaturation.
[8] The medical device according to [2], wherein
   the generation unit is further configured to generate a display image to be displayed in a different mode for each type of the output mode imparted by the output mode information imparting unit.
[9] The medical device according to [2], wherein
   the fluorescence indicates a trajectory of a distal end of the energy device.
[10] The medical device according to [2], wherein
   the output mode includes a first mode and a second mode different from the first mode, and
   the generation unit is further configured to generate a display image in which only the fluorescence region to which information of the first mode is imparted is displayed in a highlighted manner as the output mode, and a display image in which only the fluorescence region to which information of the second mode is imparted is displayed in a highlighted manner as the output mode.
[11] The medical device according to [10], wherein
   the generation unit is further configured to generate, in fluorescence image, a display image to be displayed in a different mode for each type of the output mode imparted by the output mode information imparting unit.
[12] The medical device according to [10], wherein
   the generation unit is further configured to generate, in a white light image, a display image to be displayed in a different mode for each type of the output mode imparted by the output mode information imparting unit.
[13] The medical device according to [1], wherein
   the output mode information imparting unit is further configured to impart the information of the output mode of the energy device to a region exceeding a specific fluorescence intensity among fluorescence regions in the fluorescence image.
[14] A medical system comprising:
   an imaging device configured to capture an image of a subject;
   a light source device configured to emit excitation light that excites a substance generated by applying a thermal treatment to a biological tissue; and
   a control device electrically connected to an imaging device and configured to communicate with a device control device that controls an energy device that cauterizes a treatment target, the control device comprising:
   an output mode information imparting unit configured to impart, based on output information of the energy device, information of an output mode of the energy device to a fluorescence region in a fluorescence image based on fluorescence generated by excitation light that excites a substance generated by cauterization using the energy device; and
   an output unit configured to output information in which the information of the output mode is imparted to the fluorescence region by the output mode information imparting unit.
[15] An operation method of a medical device, the operation method being executed by the medical device, the operation method comprising:
   imparting, by an output mode information imparting unit, based on output information of an energy device, information of an output mode of the energy device to a fluorescence region in a fluorescence image based on fluorescence generated by excitation light that excites a substance generated by cauterization using the energy device; and
   outputting, by an output unit, information in which the information of the output mode is imparted to the fluorescence region by the output mode information imparting unit.
[16] A non-transitory computer-readable recording medium with an executable program stored thereon, the program causing a medical device to execute:
   generating a fluorescence image based on excitation light that excites a substance generated by cauterization using an energy device and fluorescence generated by the excitation light;
   imparting, based on output information of the energy device, information of an output mode of the energy device to a fluorescence region in the fluorescence image based on the fluorescence generated by the excitation light that excites the substance generated by cauterization using the energy device; and
   outputting information in which the information of the output mode is imparted to the fluorescence region in the imparting.
[17] A medical device comprising a processor comprising hardware, the processor being configured to:
   extract a thermal treatment region of an energy device from a fluorescence image for a biological tissue;
   impart information of an output mode of the energy device to the thermal treatment region of the fluorescence image; and
   output information in which the information of the output mode is imparted to the thermal treatment region.
[18] The medical device according to [17], wherein the processor is further configured to generate the fluorescence image.
[19] The medical device according to [17], wherein
   the output mode is either an incision mode in which a treatment target is incised or a sealing mode in which a treatment target is coagulated and sealed.
[20] The medical device according to [17], wherein the processor is further configured to:
   extract thermal treatment regions from a first fluorescence image and a second fluorescence image, the thermal treatment regions being generated by different output modes of the energy device;
   determine presence or absence of a new thermal treatment region present only in the second fluorescence image for the extracted thermal treatment regions;
   impart information of the different output modes to the new thermal treatment region and a thermal treatment region other than the new thermal treatment region.
[21] The medical device according to [20], wherein
   the second fluorescence image is captured later in time than the first fluorescence image.
[22] The medical device according to [17], wherein
   the new thermal treatment region is a fluorescence region of a substance generated by cauterizing the biological tissue with the energy device.
[23] The medical device according to [22], wherein
   the substance is an advanced glycation end-product generated by thermal denaturation.
[24] The medical device according to [18], wherein
   the processor is further configured to generate a display image to be displayed in a different mode for each type of the output mode.
[25] The medical device according to [18], wherein
   the thermal treatment region indicates a trajectory of a distal end of the energy device.
[26] The medical device according to [18], wherein
   the output mode includes a first mode and a second mode different from the first mode, and
   the processor is further configured to generate a display image in which only the thermal treatment region to which information of the first mode is imparted is displayed in a highlighted manner as the output mode, and a display image in which only the thermal treatment region to which information of the second mode is imparted is displayed in a highlighted manner as the output mode.
[27] The medical device according to [26], wherein
the processor is further configured to generate, in fluorescence image, a display image to be displayed in a different mode for each type of the output mode.
[28] The medical device according to [26], wherein
   the processor is further configured to generate, in a white light image, a display image to be displayed in a different mode for each type of the output mode.
[29] The medical device according to [17], wherein
   the processor is further configured to impart the information of the output mode of the energy device to a region exceeding a specific fluorescence intensity among thermal treatment regions in the fluorescence image.
[30] The medical device according to [17], wherein the processor is further configured to extract the thermal treatment region by executing contour extraction based on a luminance value.
[31] The medical device according to [20], wherein the processor is further configured to:
   acquire the information of the different output modes of the energy device when it is determined that the new fluorescence region is present only in the second fluorescence image; and
   impart the acquired information of the different output modes to the new thermal treatment region and the thermal treatment region other than the new thermal treatment region.
[32] The medical device according to [20], wherein the processor is further configured to generate a display image in which the information of the different output modes is imparted to the new thermal treatment region and the thermal treatment region other than the new thermal treatment region.
[33] The medical device according to [17], wherein the information of the output modes includes at least one of a preset hue according to the output mode or textual information indicating the output mode.
[34] A medical system comprising:
   an imaging device configured to capture an image of a subject;
   a light source device configured to emit excitation light that excites a substance generated by applying a thermal treatment to a biological tissue; and
   a control device electrically connected to an imaging device and configured to communicate with a device control device that controls an energy device that cauterizes a treatment target, the control device comprising a processor comprising hardware, the processor being configured to:
   extract a thermal treatment region of the energy device from a fluorescence image for the biological tissue;
   impart information of an output mode of the energy device to the thermal treatment region of the fluorescence image; and
   output information in which the information of the output mode is imparted to the thermal treatment region.
[35] An operation method of a medical device, the operation method comprising:
   extracting a thermal treatment region of an energy device from a fluorescence image for a biological tissue;
   imparting information of an output mode of the energy device to the thermal treatment region of the fluorescence image; and
   outputting information in which the information of the output mode is imparted to the thermal treatment region.
[36] A non-transitory computer-readable recording medium with an executable program stored thereon, the program causing a medical device to execute:
   generating a fluorescence image based on excitation light that excites a substance generated by cauterization using an energy device and fluorescence generated by the excitation light;
   extracting a thermal treatment region of the energy device from the fluorescence image;
   imparting information of an output mode of the energy device to the thermal treatment region of the fluorescence image; and
   outputting information in which the information of the output mode is imparted to the thermal treatment region.

What is claimed is:

1. A control device for use with a medical device comprising a processor comprising hardware, the processor being configured to:
extract a first thermal treatment region treated by an energy device from a first fluorescence image for a biological tissue;
impart information of an output mode of the energy device to the first thermal treatment region of the fluorescence image; and
output the information of the output mode imparted to the first thermal treatment region.

2. The control device according to claim 1, wherein the processor is further configured to generate the fluorescence image.

3. The control device according to claim 1, wherein
the output mode is either an incision mode in which a treatment target is incised or a sealing mode in which a treatment target is coagulated and sealed.

4. The control device according to claim 1, wherein
the processor is further configured to:
extract second thermal treatment regions from a second fluorescence image, the first and the second thermal treatment regions being generated by different output modes of the energy device;
determine presence or absence of a third thermal treatment region present only in the second fluorescence image for the extracted first and second thermal treatment regions;
impart information of the different output modes to the third thermal treatment region and a fourth thermal treatment region other than the third thermal treatment region.

5. The control device according to claim 4, wherein
the second fluorescence image is captured later in time than the first fluorescence image.

6. The control device according to claim 1, wherein
the first thermal treatment region is a fluorescence region of a substance generated by cauterizing the biological tissue with the energy device.

7. The control device according to claim 6, wherein
the substance is an advanced glycation end-product generated by thermal denaturation.

8. The control device according to claim 2, wherein
the processor is further configured to generate a display image to be displayed in a different mode for each type of the output mode.

9. The control device according to claim 2, wherein
the first thermal treatment region indicates a trajectory of a distal end of the energy device.

10. The control device according to claim 2, wherein
the output mode includes a first mode and a second mode different from the first mode, and
the processor is further configured to generate one or more of:
a first display image in which only the first thermal treatment region to which information of the first mode is imparted is displayed in a highlighted manner as the output mode, and
a second display image in which only a second thermal treatment region to which information of the second mode is imparted is displayed in a highlighted manner as the output mode.

11. The control device according to claim 10, wherein
the processor is further configured to generate, in fluorescence image, a third display image to be displayed in a different mode for each type of the output mode.

12. The control device according to claim 10, wherein
the processor is further configured to generate, in a white light image, a third display image to be displayed in a different mode for each type of the output mode.

13. The control device according to claim 1, wherein
the processor is further configured to determine a region exceeding a specific fluorescence intensity among thermal treatment regions in the fluorescence image to impart the information.

14. The control device according to claim 1, wherein
the extracted first thermal treatment region is extracted contour based on a luminance value.

15. The control device according to claim 4, wherein
the processor is further configured to acquire the information of the different output modes when it is determined that the new fluorescence region is present only in the second fluorescence image so as to impart the acquired information to the third thermal treatment region and the fourth thermal treatment region.

16. The control device according to claim 4, wherein
the processor is further configured to generate a display image in which the information of the different output modes is imparted to the third thermal treatment region and the fourth thermal treatment region.

17. The control device according to claim 1, wherein
the information of the output modes includes at least one of a preset hue according to the output mode or textual information indicating the output mode.

18. A medical system comprising:
an imaging device configured to capture an image of a subject;
a light source device configured to emit excitation light that excites a substance generated by applying a thermal treatment to a biological tissue; and
the control device according to claim 1, wherein the control device is electrically connected to an imaging device and configured to communicate with a device control device that controls an energy device that cauterizes a treatment target.

19. An operation method of a medical device, the operation method comprising:
extracting a thermal treatment region treated by an energy device from a fluorescence image for a biological tissue;
imparting information of an output mode of the energy device to the thermal treatment region of the fluorescence image; and
outputting the information of the output mode imparted to the thermal treatment region.

20. A non-transitory computer-readable recording medium with an executable program stored thereon, the program causing a medical device to execute:
extracting a thermal treatment region treated by an energy device from the fluorescence image for a biological tissue;
imparting information of an output mode of the energy device to the thermal treatment region of the fluorescence image; and
outputting the information of the output mode is imparted to the thermal treatment region.
